(19) United States
(12) Patent Application Publication
(10) Pub. No.: US 2026/0241200 A1
(43) Pub. Date: Aug. 20, 2026

(54) COMPUTER VISION-BASED MONITORING SYSTEM FOR JAUNDICE TREATMENT

(71) Applicant: ATENEO DE MANILA UNIVERSITY, Quezon City, Metro Manila (PH)

(72) Inventors: Paul M. CABACUNGAN, Quezon City (PH); Paul Ryan A. SANTIAGO, Quezon City (PH); Bryan Kristofer A. MANABAT, Quezon City (PH); Isaiah Dale L. DELOS REYES, Quezon City (PH); Carlos M. OPPUS, Quezon City (PH); Nathaniel Joseph C. LIBATIQUE, Quezon City (PH); Gregory L. TANGONAN, Quezon City (PH)

(73) Assignee: ATENEO DE MANILA UNIVERSITY, Quezon City, Metro Manila (PH)

(21) Appl. No.: 18/852,362

(22) PCT Filed: Sep. 11, 2024
(86) PCT No.: PCT/PH2024/050011

§ 371 Date: Sep. 27, 2024

(30) Foreign Application Priority Data
Sep. 11, 2023 (PH) 12023050495

(51) Int. Cl. A61N 5/06; A61B 5/00; A61B 5/103

(52) U.S. Cl. CPC A61N 5/0621; A61B 5/0013; A61B 5/0022; A61B 5/0077; A61B 5/1032; A61B 5/443; A61B 5/4836; A61B 5/74; A61N 2005/0626

(57) ABSTRACT

The present invention relates to a computer-based jaundice monitoring system that employs image processing techniques to monitor and detect jaundice in patients. The system includes a camera for capturing images of the patient, a computing device for image processing and analysis, a local communication network for interconnecting system components, and a light panel integrated with the camera to provide phototherapy. The computing device identifies the patient's skin region, analyzes dominant colors within the skin region, detects yellow pigmentation indicative of jaundice, and generates notifications accordingly.

### TECHNICAL FIELD OF THE INVENTION

The present invention relates to patient monitoring systems. More particularly, the present invention relates to computer vision-based monitoring system for jaundice treatment.

### BACKGROUND OF THE INVENTION

Neonatal jaundice, a common condition in newborns caused by excess bilirubin leading to yellowing of the skin and eyes. Recent research indicates its widespread occurrence, particularly in low- and middle-income nations. Risk factors like prematurity and low birth weight are noted. Early detection and treatment are crucial to prevent severe outcomes. Phototherapy, using blue light, is a standard treatment, but traditional systems have limitations. LED-based phototherapy is more cost-effective and efficient. Computer vision technology has emerged as a promising solution for improving jaundice management, particularly in resource-limited settings. By using computer vision algorithms to analyze skin color, doctors and healthcare workers can accurately diagnose and monitor jaundice without relying on expensive equipment or specialized training. This technology can also enable the development of low-cost, portable phototherapy lights that can be easily deployed in areas with limited access to healthcare. The invention aims to contribute to the improvement of healthcare solutions for neonates by providing an affordable, accessible, and effective phototherapy device that can be used in low-resource settings. By combining phototherapy treatment with non-invasive skin color monitoring, the invention has the potential to improve the accuracy and effectiveness of jaundice treatment and reduce the risks and complications associated with untreated jaundice.

Different patient monitoring systems are currently disclosed in the art. For instance, Hashim et al. (2021) discloses a neonatal jaundice detection system by detecting the skin area by converting the image to the Lab color space and getting the values of the B channel and Cb channel, these values are compared with the predefined threshold value. If the value of either B or Cb channel is less than the threshold, the diagnosis result is “Jaundice” and the infant needs phototherapy. Otherwise, the diagnosis result is “Normal”.

KR101623167B1 discloses a monitoring system for a newborn baby. The system comprises a camera with face recognition and body recognition modules. The system also comprises a facial color discriminating module which can discriminate jaundice and operates an emergency module when jaundice is detected.

However, these patient monitoring systems do not disclose the patient monitoring system as shown by the present invention.

### SUMMARY OF THE INVENTION

The object of the present invention is to provide a system for monitoring jaundice.

Accordingly, the present invention provides a computer vision-based jaundice monitoring system, comprising:
a. a camera configured to capture an image of a patient;
b. a computing device in communication with the camera, the computing device is programmed to process the captured image, the processing comprising:
   i. identifying the patient's skin region within the captured image,
   ii. analyzing the dominant colors present in the patient's skin region,
   iii. determining the presence of yellow pigmentation based on the dominant colors, and
   iv. generating and transmitting a notification upon detection of yellow pigmentation
c. a light panel integrated with the camera for administering phototherapy to the patient; and
d. a local communication network facilitating connectivity between the camera, the computing device, and the light panel.

### BRIEF DESCRIPTION OF THE FIGURES

FIG. 1 illustrates a computer vision-based jaundice monitoring system according to a preferred embodiment of the present invention;

FIG. 2 illustrates a method for monitoring jaundice using a computer-based system according to a preferred embodiment of the present invention.

### DETAILED DESCRIPTION OF THE INVENTION

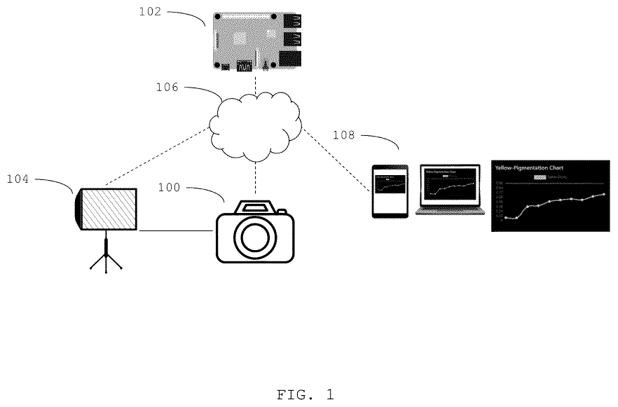

FIG. 1 illustrates a computer vision-based jaundice monitoring system according to a preferred embodiment of the present invention. The system comprises a camera 100 configured to capture an image of a patient. A computing device 102 in communication with camera 100 processes the captured image. The image processing comprises identifying the patient's skin region within the captured image. Next, the computing device 102 analyzes the dominant colors present in the patient's skin region. Then, the presence of yellow pigmentation is determined based on the dominant colors. Lastly, upon determination of yellow pigmentation, the computing device 102 generates and transmits a notification.

The system further comprises a light panel 104 integrated with the camera 100. The light panel 104 provides phototherapy to the patient.

Further, the system comprises a local communication network 106, facilitating connectivity between the camera 100, computing device 102, and light panel 104. Preferably, the local communication network 106 is a Wi-Fi network. Alternatively, local communication network 106 is a Bluetooth network or other communication network technology available in the art.

In a preferred embodiment, the computing device 102 sends a signal for the controlled intensity of the light panel 104 based on the yellow pigmentation of the patient's skin.

In another further embodiment, a monitoring device 108 is further connected to the local communication network 106. The monitoring device 108 receives notification and data from the computing device 102.

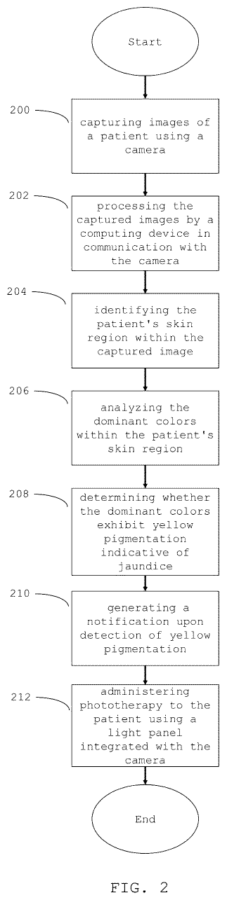

FIG. 2 illustrates a method for monitoring jaundice using a computer-based system according to a preferred embodiment of the present invention. The method starts with capturing images of a patient using a camera (step 200). Then, a computing device, in communication with the camera, processes the captured images (step 202). The process comprises identifying the patient's skin region within the captured image (step 204). Preferably, the identification is done by identifying pixels as skin using the following rules:

$$\begin{matrix}( i ) & \\ R > 95 and G > 40 and B > 20 & ( 1 )\end{matrix}$$

$$\begin{matrix}( ii ) & \\ Max ( R , G , B ) - Min ( R , G , B ) > 15 & \end{matrix}$$

$$\begin{matrix}( iii ) & \\ \vert R - G \vert > 15 & \end{matrix}$$

$$\begin{matrix}( iv ) & \\ R > G and R > B & \end{matrix}$$

where R is the red channel, G is the green channel, and B is the blue channel.

The first rule defines the range of RGB values, the second rule describes the range of saturation, the third rule specifies the acceptable difference of the red and green channels, and the fourth rule necessitates that the red channel is greater than the green and blue channels. If all these rules are satisfied, a pixel is considered a skin pixel.

Next, the computing device analyzes the dominant colors within the patient's skin region (step 206). This is done via k-means clustering.

Then, the computing device determines whether the dominant colors exhibit yellow pigmentation (step 208). The b* value of a color on the CIE L*a*b color space is used as the key variable. CIE L*a*b* is device-independent, 3D color space that enables accurate measurement and comparison of all perceivable colors expressed in three values: L* indicating perceptual lightness and a* and b* as chromaticity coordinates.

The yellow score is determined by the weighted average of the extracted dominant skin colors' normalized b* coordinate relative to a reference yellow point and a reference “normal” skin point. This is done to provide a more interpretable representation of data for monitoring purposes. The formula is given by:

$$Yellow Score = {\sum }_{i} wi ( bi - b min ) / ( b max - b min )$$

Where:
bi is the b*-coordinate of the extracted color i's position in the CIE L*a*b* color space;
bmin is the b*-coordinate of a reference normal skin pixel;
bmax is the b*-coordinate of a reference maximum yellow point;
wi is the weight assigned to each extracted color i
determined by the number of pixels clustered around it (how dominant it is).

Afterwards, the computing device generates a notification upon detection of yellow pigmentation (step 210).

Also, the computing device sends a signal for controlling the intensity of a light panel, thereby administering phototherapy to the patient (step 212).

In a further embodiment of the invention, the computing device utilizes machine learning algorithms for improved accuracy in determining yellow pigmentation.

It is contemplated for embodiments described herein to extend to individual elements and concepts described herein, independently of other concepts, ideas or system, as well as for embodiments to include combinations of elements recited anywhere in this application. It is to be understood that the invention is not limited to the embodiments described in detail herein with reference to the accompanying drawings. As such, many variations and modifications will be apparent to practitioners skilled in this art. Illustrative embodiments such as those depicted refer to a preferred form but is not limited to its constraints and is subject to modification and alternative forms. Accordingly, it is intended that the scope of the invention be defined by the following claims and their equivalents. Moreover, it is contemplated that a feature described either individually or as part of an embodiment may be combined with other individually described features, or parts of other embodiments, even if the other features and embodiments make no mention of the said feature. Hence, the absence of describing combinations should not preclude the inventor from claiming rights to such combinations.

1. A computer vision-based jaundice monitoring system, comprising:
a. a camera configured to capture an image of a patient;
b. a computing device in communication with the camera, the computing device is programmed to process the captured image, the processing comprising:
i. identifying the patient's skin region within the captured image,
ii. analyzing the dominant colors present in the patient's skin region,
iii. determining the presence of yellow pigmentation based on the dominant colors, and
iv, generating and transmitting a notification upon detection of yellow pigmentation;
c. a light panel integrated with the camera for administering phototherapy to the patient; and
d. a local communication network facilitating connectivity between the camera, the computing device, and the light panel.

2. The system in claim 1, further comprising a monitoring device, connected the local communication network, for receiving the notification and patient data.

3. The system in claim 1, wherein the computing device utilizes machine learning algorithms for improved accuracy in determining yellow pigmentation.

4. The system in claim 1, wherein the local communication network is a wireless network utilizing Wi-Fi or Bluetooth technology.

5. The system in claim 1, wherein the light panel is adjustable in intensity to provide controlled phototherapy to the patient.

6. A method for monitoring jaundice using a computer-based system, comprising the steps of:
a. capturing images of a patient using a camera;
b. processing the captured images by a computing device in communication with the camera, the processing comprising:
i. identifying the patient's skin region within the captured image,
ii. analyzing the dominant colors within the patient's skin region,
iii. determining whether the dominant colors exhibit yellow pigmentation indicative of jaundice, and
iv. generating a notification upon detection of yellow pigmentation; and
c. administering phototherapy to the patient using a light panel integrated with the camera.
